(19) United States
(12) Patent Application Publication
(10) Pub. No.: US 2024/0020440 A1
(43) Pub. Date: Jan. 18, 2024

(54) METHOD FOR DIRECTED DESIGN AND REGULATION OF FERMENTED FOOD BASED ON FLAVOR GROUP AND ITS APPLICATION

(71) Applicant: Jiangnan University, Wuxi (CN)

(72) Inventors: Qun WU, Wuxi (CN); Yan XU, Wuxi (CN); Rubing DU, Wuxi (CN)

(21) Appl. No.: 18/462,508

(22) Filed: Sep. 7, 2023

(30) Foreign Application Priority Data
Apr. 23, 2023 (CN) 202310441788.9

(51) Int. Cl. G06F 30/20; C12G 3/02; C12Q 1/04; C12N 1/16; C12N 1/18

(52) U.S. Cl. CPC G06F 30/20; C12G 3/02; C12Q 1/04; C12N 1/165; C12N 1/185; C12R 2001/645

(57) ABSTRACT

The present disclosure discloses a method for directed design and regulation of fermented food based on flavor group and its application, belonging to the fields of biology and food technology. The method involves the analysis of a significant number of fermented food samples, resulting in the identification of 20 core flavor components and six highly productive functional microorganisms associated with these core flavor components. Mathematical models have been constructed based on the correlation between the core flavor components and the microbial community structure. The method allows for the directed design and regulation of flavor components during the production of fermented food, ensuring stable production of fermented foods.

### REFERENCE TO SEQUENCE LISTING

The instant application contains a Sequence Listing in XML format as a file named “3054-PC230001A.xml”, created on Aug. 24, 2023, of 24 kB in size, and which is hereby incorporated by reference in its entirety.

### TECHNICAL FIELD

The disclosure relates to a method and its application for targeted design and regulation of fermented food based on flavor profiles, and belongs to the fields of biotechnology and food technology.

### BACKGROUND

Fermented foods play a crucial role in human dietary structure, occupying a significant position. These foods are rich in nutritional components and possess unique flavors. The flavor characteristics are primarily determined by the profile of core flavor compounds (composition and structure of flavor components), which are essential factors influencing consumer preferences and consumption tendencies. Additionally, there is an increasing demand for diversity in the flavor characteristics of fermented foods. Achieving targeted design and regulation of the flavor compound profile in fermented foods is crucial to ensure product quality stability and to meet the diverse needs of consumers.

The flavor components of fermented foods are primarily generated during the metabolic processes of microbial communities in the fermentation. However, most fermented foods undergo a natural fermentation process, where the microbial communities involved are influenced by various human and natural factors. This results in an unstable microbial community structure, leading to inconsistent product quality. Currently, in different fermented foods, the use of synthetic microbial communities as replacements for natural microbial communities has enabled the reproduction of product flavor components. In the fermentation of bean paste, JIA and his team (Jia Y, Niu C T, Lu Z M, Zhang X J, Chai L J, Shi J S, Xu Z H, Li Q. 2020. A bottom-up approach to develop simplified microbial community model with desired functions: Application for efficient fermentation of broad bean paste with low salinity. Appl Environ Microbiol 86: e00306-20) selected Aspergillus oryzae, Bacillus subtilis, Staphylococcus gallinarum, Weissella confusa, and Zygosaccharomyces rouxii as initial fermenting agents. These effectively recreated the flavor metabolizing functions of the in-situ brewed microbial communities. In the fermentation process of Chinese liquor, WANG (Wang SL, Wu Q, Nie Y, Xu Y. 2019. Construction of synthetic microbiota for reproducible flavor metabolism in Chinese light aroma type liquor produced by solid-state fermentation. Appl Environ Microbiol 85:e03090-18) selected Lactobacillus acetotolerans, Pichia kudriavzevii, Geotrichum candidum, Candida vini, and Saccharomyces cerevisiae as the initial fermentation agents, thus replicating the metabolic characteristics of flavor components of the indigenous microbial community during in-situ brewing. However, there is still no available method to achieve targeted design and regulation of the flavor component profile in fermented foods currently. The difficulties lie in the following aspects: (1) the complex composition of flavor components in fermented foods; (2) the unclear correspondence between the flavor component profile of fermented foods and the microbial structure.

### SUMMARY

In this disclosure, a method and its application for the targeted design and regulation of fermented food based on flavor profiles are provided, addressing at least one of the following technical problems: (1) Inadequate understanding of the essential flavor components in fermented foods; (2) Insufficient availability of microorganisms capable of producing high yields of core flavor components; (3) Unclear correspondence between the microbial community structure used for food fermentation and the content of individual flavor components in the fermented product; (4) Unclear correspondence between the microbial community structure used for food fermentation and the flavor component profile in the fermented product; (5) Inability to achieve targeted control of the flavor component profile in fermented foods.

The disclosure provides a method for analyzing the core flavor components in fermented food samples. This method includes detecting volatile components in N fermented food samples, where the volatile components in at least one type of fermented food with an Odor Activity Value (OAV) greater than or equal to 1, and a distribution frequency among samples greater than or equal to 70% are identified as core flavor components. In this method, N should be equal to or greater than 56.

In an implementation, the fermented food samples are selected from multiple different regions.

In an implementation, N is equal to 80.

In an implementation, the core flavor components are determined through detection and statistical analysis of 167 volatile components in 80 fermented food samples, which are collected from 14 cities nationwide. In these 80 samples, a total of 167 volatile components are identified.

In an implementation, the core flavor components are identified as volatile components with an Odor Activity Value (OAV) greater than or equal to 1 and a distribution frequency among samples greater than or equal to 70% in at least one type of fermented food.

In an implementation, the core flavor components consist of 20 different compounds, including 5 alcohol compounds, 6 acid compounds, and 9 ester compounds. Specifically, the alcohol compounds are 2-methyl-1-propanol, 1-butanol, 3-methyl-1-butanol, 1-hexanol, and phenylethyl alcohol. The acid compounds are acetic acid, 3-methyl-butanoic acid, pentanoic acid, hexanoic acid, octanoic acid, and nonanoic acid. The ester compounds are ethyl acetate, butanoic acid ethyl ester, acetate 3-methyl-1-butanol, pentanoic acid ethyl ester, hexanoic acid ethyl ester, heptanoic acid ethyl ester, octanoic acid ethyl ester, decanoic acid ethyl ester, and benzenepropanoic acid ethyl ester.

The disclosure further provides functional microorganisms that can efficiently synthesize core flavor components, and to construct a minimal synthetic microbial community capable of synthesizing these 20 core flavor components.

In an implementation, the functional microorganisms mentioned include 3 strains of Saccharomyces cerevisiae, 2 strains of Debaryomyces hansenii, and 1 strain of Wickerhamomyces anomalus. The specific functional microorganisms are as follows:

Saccharomyces cerevisiae CCTCC M 2023562, deposited in China Center for Type Culture Collection on Apr. 19, 2021, with an accession number of CCTCC M 2023562.

Saccharomyces cerevisiae CCTCC M 2023560, deposited in China Center for Type Culture Collection on Apr. 19, 2021, with an accession number of CCTCC M 2023560.

Saccharomyces cerevisiae CCTCC M 2023558, deposited in China Center for Type Culture Collection on Apr. 19, 2021, with an accession number of CCTCC M 2023558.

Debaryomyces hansenii CCTCC M 2023557, deposited in China Center for Type Culture Collection on Apr. 19, 2021, with an accession number of CCTCC M 2023557.

Debaryomyces hansenii CCTCC M 2023559, deposited in China Center for Type Culture Collection on Apr. 19, 2021, with an accession number of CCTCC M 2023559.

Wickerhamomyces anomalus CCTCC M 2023561, deposited in China Center for Type Culture Collection on Apr. 19, 2021, with an accession number of CCTCC M 2023561.

In an implementation, the above-mentioned 6 strains of functional microorganisms were selected from 960 yeast strains based on the genes copy number involved in flavor component synthesis and the metabolic capacity for core flavor components.

In an implementation, the 960 yeast strains are sourced from 80 fermented food samples.

In an implementation, the copy number of flavor component synthesis genes is determined using fluorescent quantitative PCR (polymerase chain reaction) technology.

In an implementation, the biomass of yeast in the fermentation samples is determined using fluorescent quantitative PCR technology.

In an implementation, the metabolic capacity of core flavor components for each yeast strain is determined using headspace solid-phase microextraction gas chromatography-mass spectrometry (HS-SPME-GC-MS) method.

In an implementation, the minimal synthetic microbial community consists of 3 strains of Saccharomyces cerevisiae, 2 strains of Debaryomyces hansenii, and 1 strain of Wickerhamomyces anomalus. Specifically, the minimal synthetic microbial community is composed of the following strains: Saccharomyces cerevisiae CCTCC M 2023562, Saccharomyces cerevisiae CCTCC M 2023560, Saccharomyces cerevisiae CCTCC M 2023558, Debaryomyces hansenii CCTCC M 2023557, Debaryomyces hansenii CCTCC M 2023559, Wickerhamomyces anomalus CCTCC M 2023561.

The disclosure further provides a method for predicting the content of specific core flavor components produced by the synthetic microbial community. This method involves using a predictive model formula to calculate the content of specific core flavor components.

In the predictive model formula, Fi=N₁+N₂×A+N₃×B+N₄×C N₅×AB+N₆×AC+N₇×BC+N₈×A²N₉×B²+N₁₀×C², where Fi represents the content of a specific flavor component, N₁ is the constant term, N₂-N₁₀ are coefficients, A represents the inoculum size of Saccharomyces cerevisiae (log₁₀ CFU/mL), B represents the inoculum size of Debaryomyces hansenii (log₁₀ CFU/mL), C represents the inoculum size of Wickerhamomyces anomalus (log₁₀ CFU/mL).

In an implementation, the synthetic microbial community is consisting of the 3 strains of Saccharomyces cerevisiae, 2 strains of Debaryomyces hansenii, and 1 strain of Wickerhamomyces anomalus, if there are multiple strains of the same yeast species, the seed cultures of different strains are first mixed in proportion based on their biomass. Then, the mixed seed cultures are inoculated into the fermentation process according to the desired inoculation ratios.

In an implementation, the specific predictive model for flavor compounds is as follows:

| | flavor | | | | | | | | | | |
|---|---|---|---|---|---|---|---|---|---|---|---|
| ID | component | N1 | N2 | N3 | N4 | N5 | N6 | N7 | N8 | N9 | N10 |
| F1 | 2-methyl- | 208.58 | −96.44 | 130.93 | −93.34 | 0 | 16.55 | 0 | 0 | −11.19 | 0 |
| | 1-propanol | | | | | | | | | | |
| F2 | 1-butanol | −0.66 | 0.02 | 0.04 | 0.34 | 0 | 0 | 0 | 0 | 0 | 0 |
| F3 | 3-methyl- | 1845.14 | 69.4 | 556.96 | 99.3 | −1.83 | 51.61 | −27.25 | −27.57 | −32.36 | −21.59 |
| | 1-butanol | | | | | | | | | | |
| F4 | 1-hexanol | 25.15 | −4.22 | 2.21 | −4.94 | −0.28 | 0.2 | −0.23 | 0.38 | 0.06 | 0.45 |
| F5 | phenylethyl | 292.98 | 169.76 | −30.31 | −47.37 | 0 | 0 | 0 | 0 | 0 | 0 |
| | alcohol | | | | | | | | | | |
| F6 | acetic | 67.61 | −17.06 | −5.3 | 17.49 | 0 | 0 | 0 | 0 | 0 | 0 |
| | acid | | | | | | | | | | |
| F7 | 3-methyl- | −3 | −0.02 | 0.32 | 1.22 | 0 | 0 | 0 | 0 | 0 | 0 |
| | butanoic | | | | | | | | | | |
| | acid | | | | | | | | | | |
| F8 | pentanoic | 191.49 | −27.11 | 0 | −27.89 | 0 | 4.52 | 0 | 0 | 0 | 0 |
| | acid | | | | | | | | | | |
| F9 | hexanoic | 132.08 | 17.13 | 0 | −44.62 | 0 | 6.39 | 0 | −4.8 | 0 | 0 |
| | acid | | | | | | | | | | |
| F10 | octanoic | 2489.93 | −310.82 | 4.94 | −409.18 | −3.92 | 54.87 | 2.78 | 0 | 0 | 0 |
| | acid | | | | | | | | | | |
| F11 | nonanoic | −14.38 | −3.75 | 26.78 | −15.93 | −0.46 | 2.43 | −0.17 | −0.74 | −1.9 | 0.19 |
| | acid | | | | | | | | | | |
| F12 | ethyl | 9327.46 | −3023.82 | 1540.05 | −887.06 | 0.17 | 448.99 | −265.82 | 0 | 0 | 0 |
| | acetate | | | | | | | | | | |
| F13 | butanoic | 14.66 | −1.95 | 0.3 | −2.98 | −0.07 | 0.14 | −0.06 | 0.13 | 0.04 | 0.19 |
| | acid ethyl | | | | | | | | | | |
| | ester | | | | | | | | | | |
| F14 | acetate 3- | 364.38 | −189.96 | 126.94 | −25.93 | 0.88 | 28.64 | −23.47 | 0 | 0 | 0 |
| | methyl-1- | | | | | | | | | | |
| | butanol | | | | | | | | | | |
| F15 | pentanoic | 7.33 | −1.21 | −0.12 | −1.21 | 0.03 | 0.07 | −0.16 | 0.06 | 0.09 | 0.14 |
| | acid ethyl | | | | | | | | | | |
| | ester | | | | | | | | | | |
| F16 | hexanoic | 571.24 | −60.79 | −24.16 | −90.12 | 1.02 | 6.17 | −3.84 | 1.47 | 4.08 | 5.14 |
| | acid ethyl | | | | | | | | | | |
| | ester | | | | | | | | | | |
| F17 | heptanoic | 1.38 | 0.13 | 0.24 | −0.36 | 0 | 0 | 0 | 0 | 0 | 0 |
| | acid ethyl | | | | | | | | | | |
| | ester | | | | | | | | | | |
| F18 | octanoic | 1253.09 | −101.86 | −36.4 | −226.52 | −4.1 | 21.49 | 10.18 | 0 | 0 | 0 |
| | acid ethyl | | | | | | | | | | |
| | ester | | | | | | | | | | |
| F19 | decanoic | 22.88 | 0.1 | 3.14 | −5.67 | 0 | 0 | 0 | 0 | 0 | 0 |
| | acid ethyl | | | | | | | | | | |
| | ester | | | | | | | | | | |
| F20 | benzene- | 0.23 | 0.19 | 0.01 | −0.14 | 0 | 0 | 0 | 0 | 0 | 0 |
| | propanoic | | | | | | | | | | |
| | acid ethyl | | | | | | | | | | |
| | ester | | | | | | | | | | |

This method has uncovered the correspondence between the structure of synthetic microbial communities used for food fermentation and the content of individual core flavor components in the fermentation product. This correspondence is a prerequisite for achieving targeted production of core flavor component profiles.

The disclosure further provides a design method for the microbial community structure that facilitates the targeted production of core flavor component profiles. This method involves the use of a mathematical model for designing the microbial community structure, where the input parameter of the mathematical model is an expected flavor component profile.

In an implementation, the calculation logic of the mathematical model is as follows: Firstly, generate several synthetic microbial community structures. Based on the predictive model of the present invention, predict the flavor component profiles corresponding to each synthetic microbial community. Then, calculate the similarity between each predicted flavor component profile and the expected flavor component profile using the Bray-Curtis similarity calculation formula. Apply a genetic algorithm to iterate and optimize the above process. When the similarity is at its maximum, the corresponding synthetic microbial community structure is considered as the optimal synthetic microbial community structure and is output as ‘a’. Simultaneously, input the optimal synthetic microbial community structure into the predictive model to obtain the corresponding flavor component profile, output as ‘b’. Calculate the similarity between output ‘b’ and the input parameter using the Bray-Curtis similarity calculation formula, and output this as ‘c’.

When there is a requirement to produce a targeted core flavor component profile, the core flavor component profile is used as the input for the mathematical model. By applying the model and the described optimization process, a synthetic microbial community that is capable of producing the desired flavor component profile can be obtained.

In an implementation, the mathematical model is used for predicting the structure of the synthetic microbial community, which can be employed for the targeted production of the core flavor component profile.

In an implementation, the Bray-Curtis similarity calculation formula is as follows:

$$Similarity = \frac{2 \times min ( \sum {s}_{a , i} , \sum {s}_{b , i} )}{\sum {s}_{a , i} + \sum {s}_{b , i}}$$

Where S_{a,i} and S_{b,i} represent the predicted and expected concentrations of flavor component “i,” respectively.

In an implementation, the mathematical model is established based on MATLAB.

In an implementation, the mathematical model is developed using MATLAB 2019a.

The disclosure further provides the application of the aforementioned mathematical model in the production of fermented foods. The application in fermented food production includes designing synthetic microbial communities for producing fermented foods with different expected core flavor component profiles. In other words, the mathematical model can be utilized to tailor the microbial community structure to achieve specific flavor characteristics in the fermented food product.

In an implementation, the expected core flavor component profile can be based on the currently detected core flavor component profile in fermented foods.

In an implementation, the expected core flavor component profile can be randomly generated.

The disclosure further provides a flavor-based method for the targeted design and regulation of fermented foods. The method includes the following steps:
(1) determining the core flavor components in the fermented food samples;
(2) screening functional microorganisms capable of synthesizing core flavor components and constructing a synthetic microbial community capable of producing core flavor components;
(3) establishing the correspondence between the structure of the synthetic microbial community and the content of individual core flavor components in the fermentation product; developing a predictive model formula that can predict the specific content of core flavor components based on the microbial community structure;
(4) combining the predictive model formula to establish a mathematical model for the targeted production of core flavor component profiles using synthetic microbial community structures;
(5) when conducting targeted design or regulation of fermented foods based on flavor profiles, identifying the desired core flavor component profile for targeted production; using the core flavor component profile as the input for the mathematical model to obtain a synthetic microbial community capable of producing that flavor component profile;
(6) utilizing the determined synthetic microbial community to produce the targeted core flavor component profile for use in the targeted design or regulation of fermented foods.

In an implementation, the core flavor components mentioned in step (1) include 5 alcohol compounds (2-methyl-1-propanol, 1-butanol, 3-methyl-1-butanol, 1-hexanol, and phenylethyl alcohol), 6 acid compounds (acetic acid, 3-methyl-butanoic acid, pentanoic acid, hexanoic acid, octanoic acid, and nonanoic acid), and 9 ester compounds (ethyl acetate, butanoic acid ethyl ester, acetate 3-methyl-1-butanol, pentanoic acid ethyl ester, hexanoic acid ethyl ester, heptanoic acid ethyl ester, octanoic acid ethyl ester, decanoic acid ethyl ester, and benzenepropanoic acid ethyl ester).

In an implementation, step (2) refers to the minimal synthetic microbial community.

In an implementation, the minimal synthetic microbial community consists of 3 strains of Saccharomyces cerevisiae, 2 strains of Debaryomyces hansenii, and 1 strain of Wickerhamomyces anomalus. Specifically, the strains are as follows: Saccharomyces cerevisiae CCTCC M 2023562, Saccharomyces cerevisiae CCTCC M 2023560, Saccharomyces cerevisiae CCTCC M 2023558, Debaryomyces hansenii CCTCC M 2023557, Debaryomyces hansenii CCTCC M 2023559 and Wickerhamomyces anomalus CCTCC M 2023561.

In an implementation, step (3) involves constructing synthetic microbial community structures with different ratios of functional microorganisms. These microbial communities are then subjected to fermentation using the initial inoculation ratios, and the content of core flavor components in the fermentation products is measured. Next, Design Expert software is utilized to build predictive models that correlate the content of each core flavor component with the synthetic microbial community structure. The independent variables in the model are the ratios of different functional microorganisms used for inoculation, while the dependent variables are the content levels of the core flavor components.

In an implementation, step (4) of the mathematical model takes an expected flavor component profile as input. The calculation logic of the mathematical model is as follows. Firstly, several synthetic microbial community structures are generated. Using the predictive model of the present invention, the flavor component profile corresponding to each synthetic microbial community is predicted. Then, the Bray-Curtis similarity calculation formula is applied to compute the similarity between each predicted flavor component profile and the expected flavor component profile. An iterative optimization process using a genetic algorithm is conducted to find the highest similarity, and the corresponding synthetic microbial community structure is considered the optimal one (output a). Simultaneously, the optimal synthetic microbial community structure is input into the predictive model to obtain the corresponding flavor component profile (output b). The Bray-Curtis similarity calculation formula is once again used to calculate the similarity between output b and the input expected flavor component profile (output c).

In an implementation, step (6) of the production process uses raw material extract as the fermentation medium, with a fermentation temperature of 30° C. and a fermentation time of 72 hours.

In an implementation, the fermentation process in step (6) takes place in 250 mL Erlenmeyer flasks, with a liquid volume of 150 mL.

In an implementation, the raw material extract used is sorghum extract.

In an implementation, the production in step (6) involves fermentative cultivation using a mixture of microbial strains, determined according to the synthetic microbial community identified in the previous step.

### Beneficial Effects

(1) In the disclosure, a large number of food fermentation samples have been conducted, leading to the identification of 20 core flavor components. Through extensive microbial screening, six strains of functional microorganisms capable of high-yield production of these 20 core flavor components when combined in fermentation were obtained, thereby constructing the minimal synthetic microbial community consisting of these six strains.

(2) In the disclosure, the relationship between the synthetic microbial community structure and the content of core flavor components have been analyzed, and a method to predict the specific content of core flavor components produced by the synthetic microbial community is developed.

(3) Additionally, the disclosure provides a flavor-based method for targeted design and regulation of fermented food products and its application. The methods disclosed can be employed to produce fermented food products with the desired core flavor component profiles, achieving targeted design and regulation of flavor profiles during the fermentation process. This enables stable production of fermented food products, meeting the diverse consumer demands for various flavors in fermented foods.

Collection of Biological Materials Saccharomyces cerevisiae CCTCC M 2023562, classified as Saccharomyces cerevisiae, was deposited in the China Center for Type Culture Collection (CCTCC) on Apr. 19, 2023, with the accession number CCTCC M 2023562.

Saccharomyces cerevisiae CCTCC M 2023560, classified named as Saccharomyces cerevisiae, was also deposited in the China Center for Type Culture Collection (CCTCC) on Apr. 19, 2023, with the accession number CCTCC M 2023560.

Saccharomyces cerevisiae CCTCC M 2023558, classified named as Saccharomyces cerevisiae, was deposited in the China Center for Type Culture Collection (CCTCC) on Apr. 19, 2023, with the accession number CCTCC M 2023558.

Debaryomyces hansenii CCTCC M 2023557, classified named as Debaryomyces hansenii, was deposited in the China Center for Type Culture Collection (CCTCC) on Apr. 19, 2023, with the accession number CCTCC M 2023557.

Debaryomyces hansenii CCTCC M 2023559, classified named as Debaryomyces hansenii, was also deposited in the China Center for Type Culture Collection (CCTCC) on Apr. 19, 2023, with the accession number CCTCC M 2023559.

Wickerhamomyces anomalus CCTCC M 2023561, classified named as Wickerhamomyces anomalus, was deposited in the China Center for Type Culture Collection (CCTCC) on Apr. 19, 2023, with the accession number CCTCC M 2023561.

### DESCRIPTION OF FIGURES

FIG. 1A shows the distribution frequency of 167 volatile components in the samples.

FIG. 1B shows the 20 identified core flavor components.

FIG. 2 shows the screening of 98 mixed samples based on fluorescent quantitative PCR, with each dot in the box plot representing the ratio of Ct value to biomass in the results of the target gene fluorescent quantitative PCR. A lower ratio indicates a higher copy number of the target gene in the unit organism.

FIG. 3A shows the minimum number of strains required for high-yield production of the 20 core flavor components.

FIG. 3B shows the phylogenetic relationship of the six high-yield strains based on 26S rDNA sequences.

FIG. 4 shows the correlation between predicted values and actual values of the content of the 20 flavor components in the predictive model.

FIG. 5 shows the model input of core flavor profile (P1) and the predicted core flavor profile (P2), where F1-F20 represent the 20 flavor components (corresponding relationships shown in Table 3). The concentration unit of each flavor component is in log₁₀ mg/L.

### DETAILED DESCRIPTION

The method for preparing the raw material extraction solution is referenced from the following literature (Kong Y, Wu Q, Zhang Y, Xu Y. 2014. In situ analysis of metabolic characteristics reveals the key yeast in the spontaneous and solid-state fermentation process of Chinese light-style liquor. Appl Environ Microbiol 80:3667-3676). The specific steps for preparing the raw material extraction solution are as follows: 2 kg of sorghum is added to 8 L of deionized water and steamed for 2 hours. Then 5 U/L of amylase is added and saccharification is carried out at 60° C. for 4 hours. The saccharification product is filtered through cheesecloth, and the filtrate is centrifuged to obtain the supernatant, with the centrifugation conditions being 8000×g for 15 minutes. The supernatant is the raw material extract. Before fermentation, the raw material extract is sterilized at 115° C. for 20 minutes.

### Example 1: Screening of Core Flavor Components in Fermented Food Samples

(1) Collection of Samples

In order to analyze the core flavor components in the fermented food samples, we collected 80 fermented food samples. These 80 samples were sourced from 14 different cities and were all samples of Chinese liquor. Specifically, the liquor samples included fragrant liquor, sauce-flavored liquor, soybean paste-flavored liquor, sesame-flavored liquor, and strong-aroma liquor. The 14 cities from which the samples were collected are as follows: Shanxi Fenyang, Henan Sanmenxia, Henan Pingdingshan), Hebei Cangzhou Shandong Weifang, Jiangsu Suqian, Anhui Xuancheng, Hubei Huangshi, Guangdong Foshan, Guizhou Renhuai, Sichuan Luzhou, Qinghai Xining, Anhui Fuyang, and Yunnan Yuxi.

(2) Detection of Volatile Components

The volatile components in the above 80 samples were analyzed using the HS-SPME-GC-MS method. The sample handling, detection conditions, and quantification method were consistent with the previous research conducted by our team (Kong Y, Wu Q, Zhang Y, Xu Y. 2014. In situ analysis of metabolic characteristics reveals the key yeast in the spontaneous and solid-state fermentation process of Chinese light-style liquor. Appl Environ Microbiol 80:3667-3676).

(3) Identification of Core Flavor Components

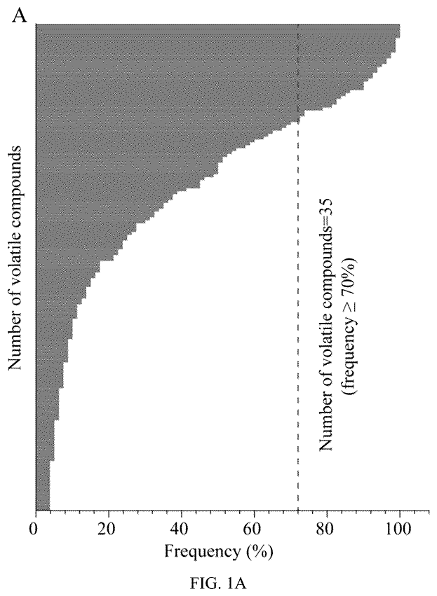

Among the 80 samples, a total of 167 volatile components were detected, with 35 volatile components having a distribution frequency of greater than or equal to 70% among the samples (FIG. 1A). Further analysis with a threshold of Odor Activity Value (OAV) greater than or equal to 1 identified 20 volatile components as core flavor compounds (FIG. 1B). These core flavor compounds include 5 alcohols (2-methyl-1-propanol, 1-butanol, 3-methyl-1-butanol, 1-hexanol, and Phenylethyl alcohol), 6 acids (acetic acid, 3-methylbutanoic acid, pentanoic acid, hexanoic acid, octanoic acid, and decanoic acid), and 9 esters (ethyl acetate, butanoic acid ethyl ester, acetate 3-methyl-1-butanol, pentanoic acid ethyl ester, hexanoic acid ethyl ester, heptanoic acid ethyl ester, octanoic acid ethyl ester, decanoic acid ethyl ester, and benzenepropanoic acid ethyl ester).

### Example 2: Screening of Functional Microorganisms for Core Flavor Components

(1) High-Throughput Screening of Strains

According to our previous research findings (Wu Q, Xu Y, Chen L. 2012. Diversity of yeast species during fermentative process contributing to Chinese Maotai-flavour liquor making. Lett Appl Microbiol 55:301-307), yeast species are the main microorganisms responsible for flavor metabolism. To screen for functional microorganisms capable of synthesizing core flavor compounds, we performed microbial screening in the aforementioned 80 fermentation food samples using three different culture media: WL, YPD, and the raw material extraction medium. The cultures were incubated at 30° C. for 72 hours. After the incubation period, 960 yeast strains were obtained by colony picking from the agar plates using a high-throughput colony picker (QPix 420, Molecular Devices, San Francisco, CA).

(2) 960 Yeast Strains Fermented Separately, Using Fermented Food Raw Material Extract as Fermentation Medium

To improve the screening efficiency, the fermentation broth from every 10 samples was pooled together to create 96 composite samples. These composite samples were then used for further screening under the same fermentation conditions of 30° C. for 72 hours.

(3) Primer Design for Fluorescence Quantitative PCR

Based on the biosynthetic pathways of the 20 core flavor compounds, it was determined that the biosynthesis of these compounds involves 6 genes (as shown in Table 1). To design the primers for the 6 genes, representative species from 18 common yeast genera in food fermentation processes were selected. These genera include Saccharomyces cerevisiae, Wickerhamomyces anomalus, Debaryomyces hansenii, Pichia kudriavzevii, Zygosaccharomyces bailii, Schizosaccharomyces pombe, Candida tropicalis, Saccharomycopsis fibuligera, Torulaspora delbrueckii, Hyphopichia burtonii, Trichosporon asahii, Clavispora lusitaniae, Kazachstania turicensis, Komagataella pastoris, Kodamaea ohmeri, Yarrowia alimentaria, Diutina bernali, and Cornuvesica acuminata. Gene sequences corresponding to these species were obtained from the NCBI database for primer design. Multiple primer pairs were designed for genes with significant sequence variations among different species, resulting in a total of 13 primer pairs.

TABLE 1
Six target genes and their associated flavor components and primers

| | | | | SEQ |
|---|---|---|---|---|
| | Core flavor | Primers | | ID |
| Gene | components | ID | Primer sequence (5′-3′) | NO: |
| Alcohol | 1-hexanol, 2- | P1 | F*: GGTGGHCAYGARGGTGBGGTG | 1 |
| Dehydrogenase | methyl-1- | | R*: TTGTAVACVGTRABACCAGCACA | 2 |
| 5 (EC:1.1.1.1) | propanol, 3- | P2 | F: GCACACAAGATTGGAGCAAC | 3 |
| | methyl-1- | | R: ACCCAYGAYGGTTCBTTCCAACA | 4 |
| | butanol, 1- | P3 | F: CGCCAAGATGCCTTTGATCG | 5 |
| | butanol, | | R: ACTCGCAGTTACCGCAAGAA | 6 |
| | Phenylethyl | P4 | F: GGTGGHCAYGARGGTGBGGTG | 7 |
| | alcohol | | R: AAACTCCCACAGCTGAACCC | 8 |
| Aldehyde | 3-Methyl- | P5 | F: AAGNTBGCHTTYACYGGYTCYAC | 9 |
| Dehydrogenase | butanoic acid, | | R: AARAYNAWRTKDGGDGAYTTACCACC | 10 |
| (EC:1.2.1.3) | pentanoic | P6 | F: TCATCAAAGAYAATRTTTGGAGA | 11 |
| | acid, | | R: ATYAAGAARRTTGCCTTYACTGG | 12 |
| | hexanoic | P7 | F: TTGTCCAGACCGTCACTTCG | 13 |
| | acid, octanoic | | R: CTTGGACTCCACAGACACCC | 14 |
| | acid, | P8 | F: CCAAGAAGGCTGCTGAGACA | 15 |
| | nonanoic acid | | R: TTCTGCGGGCAATGGTTTTG | 16 |
| Acetyl-CoA | acetic acid | P9 | F: GGNTGGTCHGGNTTYGGWGT | 17 |
| Acyltransferase | ethyl acetate, | | R: TCYTGNGGRAACATNGAYARRTG | 18 |
| 2 (EC:3.1.2.1) | Acetate 3- | P10 | F: AATGGCCATGCTMGRCGTATGGG | 19 |
| alcohol O- | methyl-1- | | R: TGCARAWGTTTYGATATARKTTTTG | 20 |
| acetyltransferase | butanol | P11 | F: CGACCATGGAGAAGAAGCCA | 21 |
| (EC:2.3.1.84) | | | R: TTTGGTGCTGGTGAGGACAG | 22 |
| Abhydrolase | Butanoic acid | P12 | F: TGTGCWCGTTCCAARATTAC | 23 |
| Domain | ethyl ester, | | R: CCAAAAGARMAWCCSACAGCRTA | 24 |
| Containing 2, | Pentanoic | P13 | F: AGCAGCCATGCTAACGAACT | 25 |
| Acylglycerol | acid ethyl | | R: GGACCACCAATCATGAGCCA | 26 |
| Lipase (EHT1) | ester, | | | |
| Abhydrolase | Hexanoic | | | |
| Domain | acid ethyl | | | |
| Containing 2, | ester, | | | |
| Acylglycerol | Heptanoic | | | |
| Lipase (EEB1) | acid ethyl | | | |
| | ester, | | | |
| | Octanoic acid | | | |
| | ethyl ester, | | | |
| | Decanoic acid | | | |
| | ethyl ester, | | | |
| | and | | | |
| | Benzene- | | | |
| | propanoic acid | | | |
| | ethyl ester | | | |

*F and R represent forward and reverse primers respectively. The base codes for ambiguity are as follows: M = A/C, R = A/G, Y = C/T, W = A/T, S = C/G, K = T/G, V = A/G/C, B = T/C/G, H = A/T/C, D = A/T/G, N = A/T/C/G

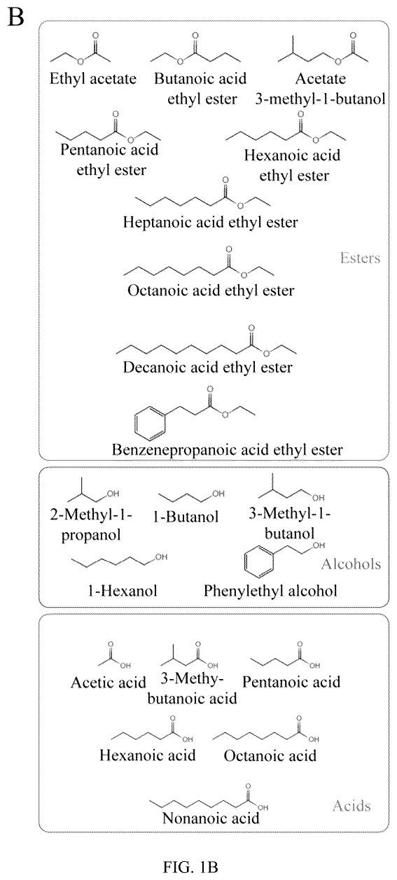

(4) Thirteen primer pairs were used in conjunction with fluorescence quantitative PCR to detect the copy numbers of biosynthetic genes for each core flavor compound in the mixed samples. DNA extraction from the mixed samples was performed using the phenol-chloroform method. The yeast biomass in the mixed samples was determined using the method described in the reference (Fierer N, Jackson JA, Vilgalys R, Jackson RB. 2005. Assessment of soil microbial community structure by use of taxon-specific quantitative PCR assays. Appl Environ Microbiol 71:4117-4120). Gene copy numbers were represented by Ct values, with a negative correlation between Ct values and gene copy numbers. The mixed samples with the highest number of gene copies per unit yeast biomass (i.e., the smallest Ct value-to-biomass ratio) were retained for further screening. As shown in FIG. 2, a total of 3 mixed samples were obtained after the secondary screening.

(5) The 30 strains of yeast from the 3 mixed samples were individually cultured in the original raw material extraction medium under the conditions of 30° C. and 72 hours.

After fermentation, 10 mL of fermentation broth was taken for flavor component analysis. The fermentation broth was centrifuged at 12,000×g and 4° C. for 10 minutes, and the supernatant was used for flavor component analysis. The detection method for flavor components was the same as described in Example 1. Out of the 30 strains, 28 strains showed normal growth. The content of flavor components among the 28 strains was normalized to a scale of 0 to 1, with a threshold of 0.7 used to determine whether a strain is a high producer. As a result, 27 strains of yeast were identified as high producers for one or more flavor compounds.

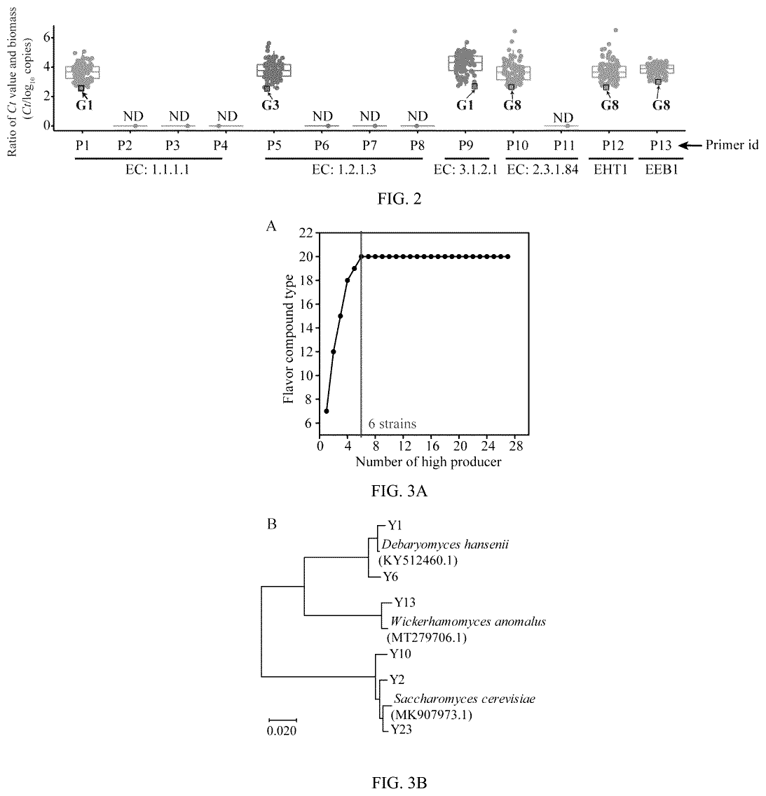

(6) To facilitate production applications, a minimal number of strains capable of high production of all 20 core flavor compounds were selected to construct the synthetic microbial community. Six strains of yeast were chosen for this purpose (FIG. 3A). Through taxonomic identification, these six strains were classified as follows: 3 strains of Saccharomyces cerevisiae, 2 strains of Debaryomyces hansenii, and 1 strain of Wickerhamomyces anomalus (FIG. 3B). Specifically, these six strains are:

Saccharomyces cerevisiae CCTCC M 2023562, classified as Saccharomyces cerevisiae, was deposited in the China Center for Type Culture Collection (CCTCC) on Apr. 19, 2023, with the accession number CCTCC M 2023562;

Saccharomyces cerevisiae CCTCC M 2023560, classified named as Saccharomyces cerevisiae, was also deposited in the China Center for Type Culture Collection (CCTCC) on Apr. 19, 2023, with the accession number CCTCC M 2023560;

Saccharomyces cerevisiae CCTCC M 2023558, classified named as Saccharomyces cerevisiae, was deposited in the China Center for Type Culture Collection (CCTCC) on Apr. 19, 2023, with the accession number CCTCC M 2023558;

Debaryomyces hansenii CCTCC M 2023557, classified named as Debaryomyces hansenii, was deposited in the China Center for Type Culture Collection (CCTCC) on Apr. 19, 2023, with the accession number CCTCC M 2023557;

Debaryomyces hansenii CCTCC M 2023559, classified named as Debaryomyces hansenii, was also deposited in the China Center for Type Culture Collection (CCTCC) on Apr. 19, 2023, with the accession number CCTCC M 2023559;

Wickerhamomyces anomalus CCTCC M 2023561, classified named as Wickerhamomyces anomalus, was deposited in the China Center for Type Culture Collection (CCTCC) on Apr. 19, 2023, with the accession number CCTCC M 2023561.

### Example 3: Construct the Relationship Between Synthetic Microbial Community Structure and the Content of Core Flavor Components

(1) Through the Design Expert software, 17 different synthetic microbial community structures were designed (Table 2).

(2) According to Table 2, the 3 yeast strains were combined in different initial inoculation ratios for fermentation. If there were different strains of the same yeast, their seed solutions were first mixed in proportion to the biomass and then inoculated according to the specified ratios. The fermentation was carried out using the raw material extraction as the fermentation medium, under the conditions of 30° C. for 72 hours. The fermentation was conducted in 250 ml triangular flasks with a liquid volume of 150 mL. The content of the 20 core flavor compounds in the fermentation products was determined using HS-SPME-GC-MS, with the same method as described in Example 1.

(3) Through the use of Design Expert software, predictive models were constructed for each of the 20 core flavor compounds in relation to the synthetic microbial community structure, with the independent variables being the inoculation ratios of the 3 yeast strains, and the dependent variable being the content of the core flavor compounds (as shown in Table 3).

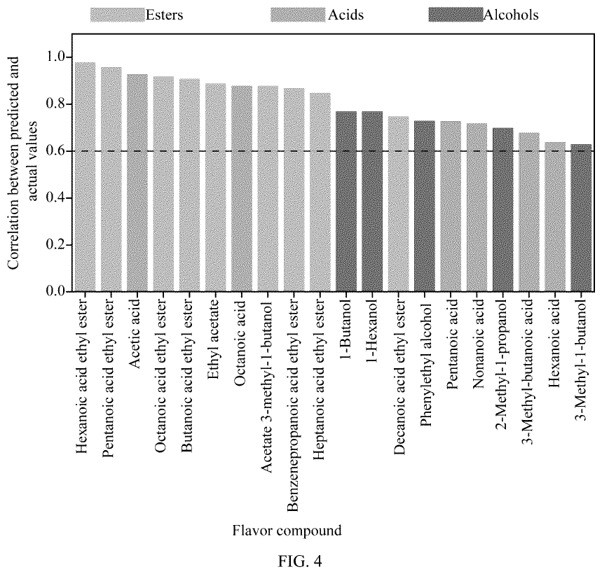

(4) Further analysis of the predictive models revealed that the predicted values obtained for the 20 core flavor compounds showed a significant positive correlation with their respective actual values (R>0.6, P<0.05), as demonstrated in FIG. 4. This indicates that the constructed predictive models for the 20 flavor compounds can accurately predict their content in the fermentation products based on the structure of the microbial community (initial inoculation ratios) consisting of the 3 yeast strains.

TABLE 2
17 different sets of synthetic microbial community structures

| Group | S. cerevisiae | D. hansenii | W. anomalus |
|---|---|---|---|
| 1 | 7 | 6 | 7 |
| 2 | 5 | 6 | 5 |
| 3 | 6 | 6 | 6 |
| 4 | 6 | 6 | 6 |
| 5 | 5 | 6 | 7 |
| 6 | 6 | 7 | 7 |
| 7 | 6 | 7 | 5 |
| 8 | 6 | 5 | 7 |
| 9 | 6 | 6 | 6 |
| 10 | 5 | 7 | 6 |
| 11 | 6 | 5 | 5 |
| 12 | 7 | 5 | 6 |
| 13 | 7 | 6 | 5 |
| 14 | 5 | 5 | 6 |
| 15 | 6 | 6 | 6 |
| 16 | 7 | 7 | 6 |
| 17 | 6 | 6 | 6 |

Noted.
The numbers in the table represent the inoculation levels in log₁₀ CFU/mL.

TABLE 3
Prediction models for 20 flavor compounds

| | flavor | | | | | | | | | | |
|---|---|---|---|---|---|---|---|---|---|---|---|
| ID | components | N1 | N2 | N3 | N4 | N5 | N6 | N7 | N8 | N9 | N10 |
| F1 | 2-methyl- | 208.58 | −96.44 | 130.93 | −93.34 | 0 | 16.55 | 0 | 0 | −11.19 | 0 |
| | 1-propanol | | | | | | | | | | |
| F2 | 1-butanol | −0.66 | 0.02 | 0.04 | 0.34 | 0 | 0 | 0 | 0 | 0 | 0 |
| F3 | 3-methyl- | 1845.14 | 69.4 | 556.96 | 99.3 | −1.83 | 51.61 | −27.25 | −27.57 | −32.36 | −21.59 |
| | 1-butanol | | | | | | | | | | |
| F4 | 1-hexanol | 25.15 | −4.22 | 2.21 | −4.94 | −0.28 | 0.2 | −0.23 | 0.38 | 0.06 | 0.45 |
| F5 | Phenylethyl | 292.98 | 169.76 | 30.31 | 47.37 | 0 | 0 | 0 | 0 | 0 | 0 |
| | alcohol | | | | | | | | | | |
| F6 | acetic | 67.61 | −17.06 | −5.3 | 17.49 | 0 | 0 | 0 | 0 | 0 | 0 |
| | acid | | | | | | | | | | |
| F7 | 3-Methyl- | −3 | −0.02 | 0.32 | 1.22 | 0 | 0 | 0 | 0 | 0 | 0 |
| | butanoic | | | | | | | | | | |
| | acid | | | | | | | | | | |
| F8 | pentanoic | 191.49 | −27.11 | 0 | −27.89 | 0 | 4.52 | 0 | 0 | 0 | 0 |
| | acid | | | | | | | | | | |
| F9 | hexanoic | 132.08 | 17.13 | 0 | −44.62 | 0 | 6.39 | 0 | −4.8 | 0 | 0 |
| | acid | | | | | | | | | | |
| F10 | octanoic | 2489.93 | −310.82 | 4.94 | −409.18 | −3.92 | 54.87 | 2.78 | 0 | 0 | 0 |
| | acid | | | | | | | | | | |
| F11 | nonanoic | −14.38 | −3.75 | 26.78 | −15.93 | −0.46 | 2.43 | −0.17 | −0.74 | 1.9 | 0.19 |
| | acid | | | | | | | | | | |
| F12 | ethyl | 9327.46 | −3023.82 | 1540.05 | −887.06 | 0.17 | 448.99 | −265.82 | 0 | 0 | 0 |
| | acetate | | | | | | | | | | |
| F13 | Butanoic | 14.66 | −1.95 | 0.3 | −2.98 | −0.07 | 0.14 | −0.06 | 0.13 | 0.04 | 0.19 |
| | acid ethyl | | | | | | | | | | |
| | ester | | | | | | | | | | |
| F14 | Acetate | 364.38 | −189.96 | 126.94 | −25.93 | 0.88 | 28.64 | −23.47 | 0 | 0 | 0 |
| | 3-methyl- | | | | | | | | | | |
| | 1-butanol | | | | | | | | | | |
| F15 | ethyl | 7.33 | −1.21 | −0.12 | −1.21 | 0.03 | 0.07 | −0.16 | 0.06 | 0.09 | 0.14 |
| | pentanoat | | | | | | | | | | |
| F16 | Hexanoic | 571.24 | −60.79 | −24.16 | 90.12 | 1.02 | 6.17 | −3.84 | 1.47 | 4.08 | 5.14 |
| | acid ethyl | | | | | | | | | | |
| | ester | | | | | | | | | | |
| F17 | Heptanoic | 1.38 | 0.13 | 0.24 | −0.36 | 0 | 0 | 0 | 0 | 0 | 0 |
| | acid ethyl | | | | | | | | | | |
| | ester | | | | | | | | | | |
| F18 | Octanoic | 1253.09 | −101.86 | −36.4 | −226.52 | −4.1 | 21.49 | 10.18 | 0 | 0 | 0 |
| | acid ethyl | | | | | | | | | | |
| | ester | | | | | | | | | | |
| F19 | Decanoic | 22.88 | 0.1 | 3.14 | −5.67 | 0 | 0 | 0 | 0 | 0 | 0 |
| | acid ethyl | | | | | | | | | | |
| | ester | | | | | | | | | | |
| F20 | Benzene | 0.23 | 0.19 | 0.01 | −0.14 | 0 | 0 | 0 | 0 | 0 | 0 |
| | propanoic | | | | | | | | | | |
| | acid ethyl | | | | | | | | | | |
| | ester | | | | | | | | | | |

Noted:
The prediction model formula is represented as follows: Fi = N₁ + N₂ × A + N₃ × B + N₄ × C+ N₅ × AB + N₆ × AC + N₇ × BC + N₈ × A² + N₉ × B² + N₁₀ × C², where Fi represents one of the flavor compounds, N₁ is the constant term, and N₂-N₁₀ are the coefficients. A represents the inoculation level of S. cerevisiae (log₁₀ CFU/mL), B represents the inoculation level of D. hansenii (log₁₀ CFU/mL), and C represents the inoculation level of W. anomalus (log₁₀ CFU/mL).

### Example 4: Establishing a Mathematical Model for Predicting the Structure of Synthetic Microbial Communities that can Directionally Produce Core Flavor Component Spectra

(1) The mathematical model was developed based on MATLAB 2019a.

(2) The input for the mathematical model is a desired flavor spectrum.

(3) The computational logic of the mathematical model is as follows: first, several synthetic microbial community structures are generated. According to the 20 prediction models in Example 3, the flavor spectra corresponding to each synthetic microbial community are predicted. Then, using the Bray-Curtis similarity calculation formula, the similarity between each predicted flavor spectrum and the desired flavor spectrum is calculated. The iterative optimization process is based on the genetic algorithm. When the similarity is highest, the corresponding synthetic microbial community structure is considered as the optimal synthetic microbial community structure and outputted as ‘a’. At the same time, the optimal synthetic microbial community structure is inputted into the 20 prediction models to get the corresponding flavor spectrum, which is outputted as ‘b’. According to the Bray-Curtis similarity calculation formula, the similarity between output ‘b’ and the input is calculated and outputted as ‘c’.

### Example 5: Based on the Mathematical Model, a Synthetic Microbial Community can be Designed to Produce a Randomly Generated Core Flavor Spectrum

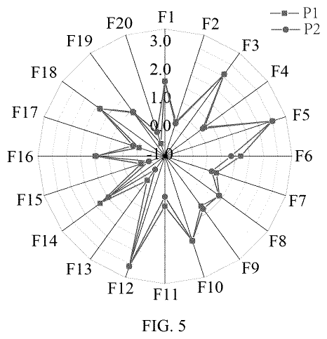

(1) The Excel “RAND” function was used to generate one randomly generated core flavor spectrum (P1). This core flavor spectrum (P1) was used as input for the mathematical model described in Example 4 to obtain the synthetic microbial community structure for producing that specific flavor spectrum. The resulting flavor spectrum is shown in FIG. 5.

(2) By using the mathematical model from Example 4, the optimal synthetic microbial community structure was calculated to be S. cerevisiae: D. hansenii: W. anomalus=10^{6.76}:10^{5.47}:10^{5.65}, with units in CFU/mL.

(3) The calculated synthetic microbial community structure from (2) was then input into the prediction models for the 20 core flavor compounds in Table 3. This resulted in the predicted flavor spectrum (P2).

(4) The similarity between the expected core flavor spectrum (P1) and the predicted flavor spectrum (P2) was calculated to be 98.54% (shown in FIG. 5). This indicates that the mathematical model is capable of designing synthetic microbial communities for the production of randomly generated core flavor spectra, enabling the qualitative design of flavor profiles in fermented foods.

Although the disclosure has been disclosed above with preferred examples, the examples are not intended to limit the disclosure. Any person familiar with this art can make various changes and modifications without departing from the spirit and scope of the disclosure. Therefore, the scope of protection of the disclosure should be as defined in the claims.

What is claimed is:

1. A method for the directed design or regulation of fermented foods based on flavor groups, characterized by the following steps:
(1) determining the core flavor components in the fermented food samples;
(2) screening for functional microorganisms that synthesize core flavor components, and building a synthetic microbial community that can synthesize core flavor components;
(3) establishing the relationship between the structure of the synthetic microbial community and the content of a single core flavor component in the fermentation products, and constructing a predictive model formula that predicts the content of a specific core flavor component through the microbial community structure;
(4) combining the predictive model formula to establish a mathematical model of the microbial community structure for the directed production of the core flavor spectrum;
(5) when designing or regulating fermented foods based on flavor groups, determining the core flavor spectrum that needs to be produced, then using this core flavor spectrum as the input for the mathematical model, and obtaining the synthetic microbial community that produces this flavor spectrum;
(6) using the synthetic microbial community determined in the previous step to produce the core flavor spectrum, which is used for the directed design or regulation of fermented foods.

2. The method according to claim 1, wherein the core flavor components in step (1) comprise 5 alcohols, 6 acids, and 9 esters; the alcohols are 2-methyl-1-propanol, 1-butanol, 3-methyl-1-butanol, 1-hexanol, and phenylethyl alcohol; the acids are acetic acid, 3-methyl-butanoic acid, pentanoic acid, hexanoic acid, octanoic acid, and nonanoic acid; the esters are ethyl acetate, butanoic acid ethyl ester, isopentyl acetate, pentanoic acid ethyl ester, hexanoic acid ethyl ester, heptanoic acid ethyl ester, octanoic acid ethyl ester, decanoic acid ethyl ester, and ethyl phenylpropionate.

3. The method according to claim 1, wherein the synthetic microbial community in step (2) refers to the minimum synthetic microbial community.

4. The method according to claim 1, wherein the synthetic microbial community in step (2) comprises 3 strains of Saccharomyces cerevisiae, 2 strains of Debaryomyces hansenii, and 1 strain of Wickerhamomyces anomalus; further, it specifically includes Saccharomyces cerevisiae CCTCC M 2023562, Saccharomyces cerevisiae CCTCC M 2023560, Saccharomyces cerevisiae CCTCC M 2023558, Debaryomyces hansenii CCTCC M 2023557, Debaryomyces hansenii CCTCC M 2023559, and Wickerhamomyces anomalus CCTCC M 2023561.

5. The method according to claim 1, wherein the mathematical model in step (4) takes as input an expected flavor profile; the calculation logic of the mathematical model is as follows: first, generating several synthetic microbial community structures and using the predictive model of the present invention to predict the flavor profile corresponding to each synthetic microbial community; then, calculating the similarity between each predicted flavor profile and the expected flavor profile using the Bray-Curtis similarity calculation formula; applying a genetic algorithm to iteratively optimize the above process, and when highest similarity achieved, considering the corresponding synthetic microbial community structure as the optimal one and becoming output ‘a’; at the same time, inputting the optimal synthetic microbial community structure into the predictive model to obtain the corresponding flavor profile as output ‘b’; the similarity between output b and the input calculating by Bray-Curtis similarity calculation formula, which becoming output ‘c’.

6. The application of core flavor compounds in the directed design or control of fermentation food, wherein the core flavor compounds comprise 5 alcohol compounds, 6 acid compounds, and 9 ester compounds; The alcohol compounds are 2-methyl-1-propanol, 1-butanol, 3-methyl-1-butanol, 1-hexanol, and Phenylethyl alcohol; the acid compounds are acetic acid, 3-methylbutanoic acid, pentanoic acid, hexanoic acid, octanoic acid, and decanoic acid; the ester compounds are ethyl acetate, Butanoic acid ethyl ester, acetate 3-methyl-1-butanol, pentanoic acid ethyl ester, hexanoic acid ethyl ester, heptanoic acid ethyl ester, octanoic acid ethyl ester, decanoic acid ethyl ester, and phenylethyl acetate.

7. The application according to claim 6, comprising the following steps:
(1) screening functional microorganisms capable of synthesizing the core flavor compounds and constructing a synthetic microbial community capable of producing the core flavor compounds;
(2) establishing a correlation between the structure of the synthetic microbial community and the content of individual core flavor compounds in the fermentation product, and developing a predictive model equation that can predict the content of specific core flavor compounds based on the microbial community structure;
(3) developing a mathematical model for the directed production of core flavor compound spectra using the predictive model equation;
(4) when conducting targeted design or regulation of fermented foods based on flavor profiles, identifying the core flavor compound spectra to be produced and using them as input for the mathematical model to obtain a synthetic microbial community for producing the desired flavor profiles;
(5) using the determined synthetic microbial community to produce the core flavor compound spectra for targeted design or regulation of fermented foods.

8. A synthetic microbial community, wherein the synthetic microbial community comprises: Saccharomyces cerevisiae CCTCC M 2023562, Saccharomyces cerevisiae CCTCC M 2023560, Saccharomyces cerevisiae CCTCC M 2023558, Debaryomyces hansenii CCTCC M 2023557, Debaryomyces hansenii CCTCC M 2023559, Wickerhamomyces anomalus CCTCC M 2023561.

9. The application of the synthetic microbial community according to claim 8 in the directed design or regulation of fermented food, wherein the application includes:
(1) establishing a correspondence between the structure of the synthetic microbial community and the content of a single core flavor component in fermented products, and constructing a prediction model formula that can predict the content of specific core flavor components through the microbial community structure;
(2) combined with the prediction model formula, establishing a mathematical model of the microbial community structure for the targeted production of core flavor component spectra;
(3) for the directed design or regulation of fermented food, determining the core flavor component spectrum that needs to be produced in a targeted manner, and then use the core flavor component spectrum as the input of the mathematical model to obtain the synthetic microbial community for producing the flavor component spectrum;
(4) using the synthetic microbial community determined in the previous step to produce the core flavor component spectrum, for the directed design or regulation of fermented food.


SEQUENCE LISTING (26 sequences)

SEQ ID NO: 1 - DNA, 21 bp, synthetic construct
  source 1..21: mol_type = other DNA
ggtgghcaygarggtgbggtg

SEQ ID NO: 2 - DNA, 23 bp, synthetic construct
  source 1..23: mol_type = other DNA
ttgtavacvgtrabaccagcaca

SEQ ID NO: 3 - DNA, 20 bp, synthetic construct
  source 1..20: mol_type = other DNA
gcacacaagattggagcaac

SEQ ID NO: 4 - DNA, 23 bp, synthetic construct
  source 1..23: mol_type = other DNA
acccaygayggttcbttccaaca

SEQ ID NO: 5 - DNA, 20 bp, synthetic construct
  source 1..20: mol_type = other DNA
cgccaagatgcctttgatcg

SEQ ID NO: 6 - DNA, 20 bp, synthetic construct
  source 1..20: mol_type = other DNA
actcgcagttaccgcaagaa

SEQ ID NO: 7 - DNA, 21 bp, synthetic construct
  source 1..21: mol_type = other DNA
ggtgghcaygarggtgbggtg

SEQ ID NO: 8 - DNA, 20 bp, synthetic construct
  source 1..20: mol_type = other DNA
aaactcccacagctgaaccc

SEQ ID NO: 9 - DNA, 23 bp, synthetic construct
  source 1..23: mol_type = other DNA
aagntbgchttyacyggytcyac

SEQ ID NO: 10 - DNA, 26 bp, synthetic construct
  source 1..26: mol_type = other DNA
aaraynawrtkdggdgayttaccacc

SEQ ID NO: 11 - DNA, 23 bp, synthetic construct
  source 1..23: mol_type = other DNA
tcatcaaagayaatrtttggaga

SEQ ID NO: 12 - DNA, 23 bp, synthetic construct
  source 1..23: mol_type = other DNA
atyaagaarrttgccttyactgg

SEQ ID NO: 13 - DNA, 20 bp, synthetic construct
  source 1..20: mol_type = other DNA
ttgtccagaccgtcacttcg

SEQ ID NO: 14 - DNA, 20 bp, synthetic construct
  source 1..20: mol_type = other DNA
cttggactccacagacaccc

SEQ ID NO: 15 - DNA, 20 bp, synthetic construct
  source 1..20: mol_type = other DNA
ccaagaaggctgctgagaca

SEQ ID NO: 16 - DNA, 20 bp, synthetic construct
  source 1..20: mol_type = other DNA
ttctgcgggcaatggttttg

SEQ ID NO: 17 - DNA, 20 bp, synthetic construct
  source 1..20: mol_type = other DNA
ggntggtchggnttyggwgt

SEQ ID NO: 18 - DNA, 23 bp, synthetic construct
  source 1..23: mol_type = other DNA
tcytgnggraacatngayarrtg

SEQ ID NO: 19 - DNA, 23 bp, synthetic construct
  source 1..23: mol_type = other DNA
aatggccatgctmgrcgtatggg

SEQ ID NO: 20 - DNA, 25 bp, synthetic construct
  source 1..25: mol_type = other DNA
tgcarawgtttygatatarkttttg

SEQ ID NO: 21 - DNA, 20 bp, synthetic construct
  source 1..20: mol_type = other DNA
cgaccatggagaagaagcca

SEQ ID NO: 22 - DNA, 20 bp, synthetic construct
  source 1..20: mol_type = other DNA
tttggtgctggtgaggacag

SEQ ID NO: 23 - DNA, 20 bp, synthetic construct
  source 1..20: mol_type = other DNA
tgtgcwcgttccaarattac

SEQ ID NO: 24 - DNA, 23 bp, synthetic construct
  source 1..23: mol_type = other DNA
ccaaaagarmawccsacagcrta

SEQ ID NO: 25 - DNA, 20 bp, synthetic construct
  source 1..20: mol_type = other DNA
agcagccatgctaacgaact

SEQ ID NO: 26 - DNA, 20 bp, synthetic construct
  source 1..20: mol_type = other DNA
ggaccaccaatcatgagcca